(19) United States
(12) Patent Application Publication
(10) Pub. No.: US 2025/0345627 A1
(43) Pub. Date: Nov. 13, 2025

(54) Device for Administering, Tracking and Reporting Light Therapy

(71) Applicant: Michael Bojkovic, Pinellas Park, FL (US)

(72) Inventor: Michael Bojkovic, Pinellas Park, FL (US)

(21) Appl. No.: 19/203,657

(22) Filed: May 9, 2025

Related U.S. Application Data
(60) Provisional application No. 63/645,032, filed on May 9, 2024.

(51) Int. Cl. A61N 5/06

(52) U.S. Cl. CPC A61N 5/0618; A61N 2005/0627; A61N 2005/0648; A61N 2005/0663

(57) ABSTRACT

A light therapy administration system is integrated with a computer device having a computer screen that enables a user to receive light therapy via the computer screen of the computer device or via a separate set of lights coupled to and controlled by the computer device while performing other tasks on the computer, such as working, interacting with one or more other applications executing on the computer device, partaking in on-line interactive calls and meetings, playing games, writing, reading, or performing any other tasks that are normally performed on a computer. In this manner, a user may receive therapeutic light as part of a light therapy session while the user is performing other tasks on the computing device administering the light therapy.

### CROSS-REFERENCE TO RELATED APPLICATION

This application is a regular filed application of and claims the benefit of priority to U.S. Provisional Application No. 63/645,032, filed May 9, 2024, entitled “Device for Administering, Tracking and Reporting Light Therapy,” the entire disclosure of which is hereby expressly incorporated by reference herein.

### FIELD OF DISCLOSURE

This patent relates to a device and method for administering light therapy to a user while enabling the user to perform other tasks and functions, including those associated with viewing and manipulating a computer or other display screen, as well as to a device and method which tracks, measures and reports the amount of light therapy provided to a user.

### BACKGROUND

Light therapy, sometimes referred to as bright light therapy (“BLT”), periodically administers light of a particular wavelength or within a particular range of wavelengths to a patient for a period of time and is known to assist in treating various disorders or conditions. The goal of BLT is to deliver an adequate amount of light through the eyes (to the retinas) of a patient to make a difference in brain functioning. The therapeutic light mimics outdoor light which is known to assist in treating or lessening the effects of various neurological conditions. For example, BLT is known to assist in the treatment of seasonal affective disorder (SAD), non-seasonal unipolar depression and non-seasonal bipolar depression, and may be helpful in treating premenstrual syndrome/premenstrual dysphoric disorder (PMS/PMDD), prenatal depression, postpartum depression, attention deficit hyperactivity disorder (ADHD), obsessive compulsive disorder (OCD), various anxiety disorders, schizophrenia, sleep deprivation, Parkinsons disease, neurodegenerative disease, and dementia. BLT has other emerging uses and can be used alone or in combination with medication to treat one or more of these conditions.

Generally speaking, light therapy operates by directing light of a certain brightness or intensity and of a certain wavelength or range of wavelengths onto or into the user's or patient's eyes for a certain amount of time each day. Depending on the condition being treated, it is generally useful to have somewhere between 5000 and 10000 lux hours delivered to a user's eyes per day with a dose of 5000 lux hours per day being considered to be most beneficial. The actual desired amount of light may depend on a number of different factors, including the condition being treated, the severity of the condition being experienced by the user, the wavelength of light being used, etc. Treatment using BLT depends on the user performing a BLT session periodically over time, with a BLT session generally being provided at least once a day and generally lasting 20 to 30 minutes, but sometimes up to one hour or more.

There are currently a number of devices that have been designed to administer light therapy, including BLT, to a user or a patient. These devices typically include a stand or a frame that holds one or more light sources which, when used properly, direct light into the user's eyes. The user must typically track or time how long the light(s) are illuminated in an effort to reach a certain desired amount of lux being incident on the user's eyes, pupils or retina. One known BLT device, commonly called a light box, includes one or more light bulbs or other sources of light mounted on a stand. The light bulb or other light source emits light of a particular wavelength and intensity. When operated, the user turns on the light bulb or other source of light and positions his or her head to face usually about 12 to 24 inches away from the light source so that the light from the light source is directed into the user's eyes. The use of a light box, however, generally precludes the user from performing other tasks that require vision, such as using a computer, walking, reading a book, etc. Moreover, the light box requires the user to track the time that the user receives light from the light source to ensure that the user receives the desired amount of light during each session.

A more sophisticated BLT administration device includes a set of light bulbs mounted onto a frame of head gear or a set of glasses, which may be placed onto the user's head. When placed on the user's head, the light bulbs mounted onto the frame direct light into the user's eyes to administer the light therapy. The lights, which may be incandescent bulbs, light emitting diodes (LEDs), or other types of illumination devices, may be mounted directly in front of the user's eyes, or may be mounted slightly above the user's eyes to direct light of an appropriate wavelength into the user's eyes for a predetermined period of time. The use of this type of BLT administration device enables the user to have some ability to perform other tasks while wearing the BLT administration device, such as using a computer positioned in front of the user, walking, reading a book, etc. However, the wavelength of the light, the brightness of the light bulbs and the positioning of the light bulbs are all dependent on the specific type of light mounted in the head gear and cannot easily be changed. Moreover, the user must still track the amount of time that the user applies the light (e.g., wears the device) to ensure that the user receives enough light per day to meet the requirements of the particular type of malady being treated, the user's physical makeup, and other factors.

As is known, currently available BLT administration devices must be worn for a particular period of time each day to administer a particular amount of light needed for the therapy. The amount of time can range from 20 minutes to over an hour, depending on condition being treated, the type of administering device, the type and brightness of the lights used in administering device and the distance between the lights and the user's eyes when the BLT is being applied. Thus, for effective treatment, a user must be positioned in front of the BLT lamp or wear the BLT glasses for a significant period of time each day. Moreover, known BLT administration devices typically prevent the user from performing other activities, or severely limit the activities that the user can perform while the BLT is being administered. For example, in many cases, known BLT administration devices cause the lights to be placed directly in front of the user's eyes which prevents the user from being able to see anything else. In other cases, the lights of the BLT administration devices are slightly offset from the user's direct line of sight, and so allow the user to see straight ahead in some limited capacity. However, the user is still limited in the activities the user can take while wearing these BLT administration device. Still further, known BLT administration devices provide a fixed amount, brightness, wavelength and positioning of the light with respect to the user and so are not very easy to alter.

### SUMMARY

A light therapy administration device or system is integrated with or made to be part of a computer device having a computer screen that enables a user to receive BLT or other light therapy via the computer screen of the computer device (or via a separate set of lights coupled to and controlled by the computer device) while performing other tasks on the computer, such as working, interacting with one or more other applications executing on the computer device, partaking in on-line interactive calls and meetings, playing games, writing, reading, or performing any other tasks that are normally performed on a computer. In this manner, a user may receive therapeutic light as part of a light therapy session while the user is performing other tasks on the computing device administering the light therapy, such as working, browsing the internet or using one or more other applications on the computing device, playing games, watching videos, participating in online communications, etc. The integrated light therapy device may take the form of a standard computing machine having a standalone or separate computer screen or display. In some cases, the integrated light therapy device may take the form of any type of extended Reality (XR) device or interface, including Augmented Reality (AR) devices, Mixed Reality (MR) devices and Virtual Reality (VR) devices, and/or may include the use of spatial computing devices such as Apple Vision Pro goggles, or may take the form of any other type of computing device with one or more display screens or display interfaces, such as a phone, a smart TV, a laptop, etc.

The light therapy device or system described herein may include a light therapy application that runs or executes on a computing device to automatically provide light therapy to the user of the computing device via a computer screen of the computing device or via a separate set of lights mounted on or mounted adjacent to the computing device and controlled by the computing device. In some cases, the light therapy device or system described herein may enable a user to select or determine the amount of light to be received during a light therapy session, the intensity or brightness (dosage) of the light, one or more wavelengths of light to be used during a light therapy session, the positioning or location of the light on the computer screen used for the light therapy, the form of the light to be received, the amount of the computer screen to be used to emit or administer the light used for the light therapy, the time or amount of lux or lux hours to be received by the user to complete a light therapy session, etc.

Moreover, in some cases, the integrated light therapy system or device described herein may track the amount of light being emitted by the computer screen or associated lights providing the therapeutic light during a therapy session, the amount of light received by the user at the user's eyes, retinas, pupils, etc., the time during which the user is exposed to the therapeutic light from the light therapy device, the wavelength or wavelengths of light used for the light therapy, etc. Moreover, the integrated light therapy device may determine when the user has received enough light for a particular light therapy session, may track and record the exposure of the user to therapeutic light over multiple light therapy sessions and may report the exposure or other details of each light therapy session to a physician or other medical advisor to assist the medical advisor in managing and overseeing the light therapy being performed for a user. Still further, the light therapy system or device described herein may administer one or more tests to the user to assist in determining if the light therapy being administered is useful or helpful, to increase or decrease the amount of light be used in each light therapy session for optimal results, or to otherwise change the specifics or parameters of the light therapy being administered to the user.

In some embodiments, the integrated light therapy device or system described herein may include one or more sensors connected to, associated with or implemented by the computer device to detect how much light is received by the user, if and how long user's eyes are open or closed during a light therapy session or at various points during a light therapy session, to measure pupil size and position with respect to a user's visual field throughout a therapy session, to accept changes by the user to change the manner of providing light to the user during a light therapy session, to change the wavelength of light provided during or for a light therapy session, to change the brightness of the light, to change the amount of surface area on the computer device that emits the light for the light therapy session, etc. In this manner, the integrated light therapy device may alter or change the type, amount, brightness, wavelength (color), visual form and/or position of the therapeutic light being administered during a particular light therapy session.

### DRAWINGS

FIG. 1 depicts an example computing system having a display screen or other electronic display implementing light therapy in addition to providing a user with other visual content.

FIG. 2 is a block diagram of an integrated light therapy device including a computing system that implements light therapy in addition to presenting visual content on a computer screen associated with one or other non-therapeutic applications, and that tracks and obtains feedback on the light therapy provided to a user.

FIG. 3 is a depiction of a set of virtual reality goggles comprising one embodiment of the integrated light therapy device described herein.

FIG. 4 is a depiction of a first embodiment of a light therapy screen display implemented using a set of virtual reality goggles.

FIG. 5 is a depiction of a second embodiment of a light therapy screen display implemented using a set of virtual reality goggles.

FIG. 6A is a depiction of a third embodiment of a light therapy screen display implemented using a set of virtual reality (VR) or (XR) goggles.

FIG. 6B is a depiction of a fourth embodiment of a light therapy screen display implemented using a set of virtual reality (VR) or (XR) goggles in which therapeutic light is provided to enhance or change portions of a visual display illustrating real information or other virtual reality information.

FIG. 7 is a depiction of an embodiment of a light therapy device implemented using a set of virtual reality goggles with a separate set of micro-LED lights mounted therein.

FIG. 8 is a depiction of an embodiment of a light therapy device including a set of virtual reality goggles with a separate set of LED lights mounted therein.

FIG. 9 is a depiction of a further embodiment of a light therapy device including a set of virtual reality goggles with multiple separate sets of LED lights mounted therein.

FIG. 10 is a depiction of an embodiment of an integrated light therapy device implemented on a computing device having a typical computer screen.

FIG. 11 is a depiction of an embodiment of an integrated light therapy device implemented on a computing device having a typical computer screen and one or more separate sets of LED lights provided as periphery devices.

FIG. 12 is a depiction of a first embodiment of a screen display used to provide light therapy on a single screen of a typical desktop computer screen.

FIG. 13 is a depiction of a second embodiment of a screen display used to provide light therapy on a single screen of a typical desktop computer screen.

FIG. 14 is a depiction of a first embodiment of a screen and display system using two screens of a typical desktop computer to provide light therapy.

FIG. 15 is a depiction of a second embodiment of a screen and display system using two screens of a typical desktop computer to provide light therapy.

FIG. 16 is a depiction of a first embodiment of an integrated light therapy device implemented on a mixed reality (MX) computing device having a single eyepiece.

FIG. 17 is a depiction of a second embodiment of an integrated light therapy device implemented on a mixed reality (MX) computing device.

FIG. 18 is a flow chart of a light therapy application including a configuration routine and a therapy implementation routine, which may be used to implement light therapy via any of the devices of FIGS. 1-17.

### DETAILED DESCRIPTION

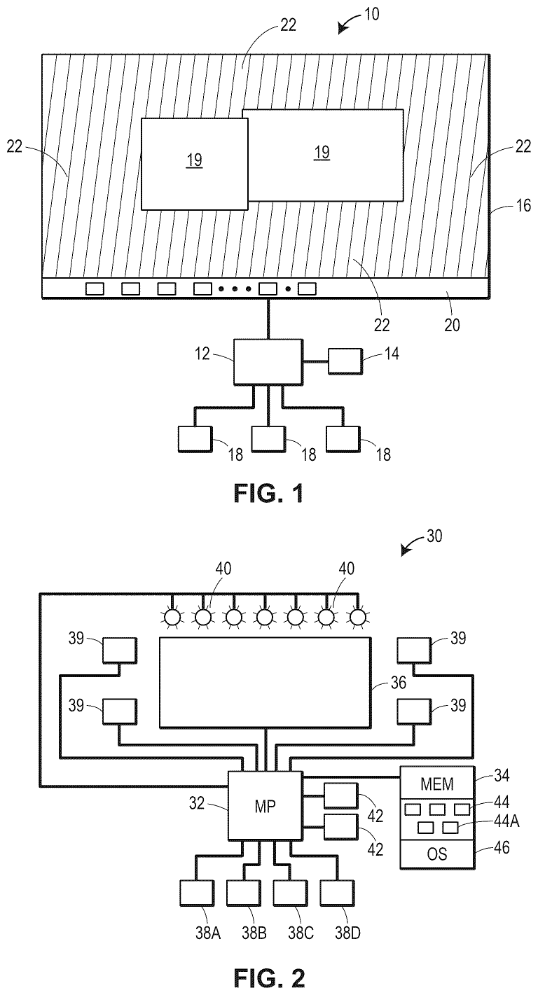

FIG. 1 depicts a computing device or system 10 that integrates a light therapy device or system, such as a BLT administration system, into a computer operating environment and, in particular, into an operating environment of a typical desktop computer system. The computing system 10 of FIG. 1 includes a processing device 12 (e.g., a microprocessor) coupled to a memory 14, to one or more computer screens or display devices 16, and to one or more input devices 18. The processing device 12 may be a general purpose microprocessor or a special purpose microprocessor programmed using any desired or known type of programming paradigm(s) and/or language(s). The input devices 18 may include any types of user input devices, such as a keyboard, a mouse, a track ball, a microphone, etc. Still further, the user display 16 may be any type of display screen, manufactured using any desired type of display technology, including light emitting diode (LED) display, organic LED (OLED) display, min-LED display, micro-LED display, plasma display, etc. technology. In this case, the computer processing device 12 implements one or more routines or applications stored in the memory 14 that execute on the computer processor 12 to provide visual content via the screen or display device 16. The routines or applications, when executed on the processor 12, may perform other actions as well, such as making sounds, controlling other peripheral devices, etc. Moreover, the input devices 18 may include sensors of any type that sense or measure one or more phenomenon and that provide signals to the processor 12 indicative of the sensed or measured phenomenon.

As illustrated in FIG. 1, the visual content provided on the screen 16 may include one or more display areas or windows 19 disposed at or on various different portions of the user display screen 16, wherein each window 19 may be associated with the same or with different applications executing on the processor 12. Additionally, an area 20 of the screen 16 may be provide visual content indicative of or provided by the operation of the operating system being implemented on the processor 12, including visual content identifying applications that are installed onto or running on the processor 12, graphical shortcuts or links to other applications or systems, links to the internet, links to one or more files, or other browsing links for locating information stored in the computer memory 14, to name but a few. As will be understood, each of the windows 19 may be associated with any desired type of application, for example, an internet browser, a word processing program, a video game, an internet communications program (such as an e-mail program, a video conferencing program, an internet telephony program, etc.) or any other program or application that may be stored in and executed by the computing system 10. Various different windows 19 may be generated by or associated with the same program or application and/or different windows 19 may be generated by or associated with different applications executing within the operating environment of the computer system 10.

Additionally, as illustrated in FIG. 1, one or more other areas 22 of the screen 16, shown in cross-hatch in FIG. 1, may be associated with a light therapy application stored in the memory 14 and executed by the computer processing device 12. In particular, the areas 22 of the screen 16 may emit light of a particular wavelength and brightness or intensity associated with or desirable for a light therapy session and thus these areas 22 emit therapeutic light that is absorbed by the user or viewer to implement a light therapy session. The therapeutic areas 22 may take up or may be disposed at any location or locations on the screen 16. Thus, while most of the therapeutic areas 22 of the screen 16 are illustrated in FIG. 1 as being on an outer edge of the screen 16, disposed generally across the entirety of the screen 16, and as taking up all of the display area of the screen 16 not associated with the windows 19 and 20, the therapeutic areas 22 could be disposed or located at any portions of the screen 16, including only at the top of the screen 16, on the sides of the screen 16, in the center of the screen 16, at regular spaced areas across the screen 16, or any combination thereof. Moreover, the therapeutic areas 22 may be symmetrical vertically, horizontally or both, or may be non-symmetrical if so desired. The therapeutic areas 22 may be partially or wholly contiguous with each other on the screen 16 or may be non-contiguous with each other and may take up or be disposed at different parts of the screen 16. The therapeutic areas 22 may be stationary on the screen 16 during a light therapy session or may move from place to place on the screen 16 in any desired manner during a light therapy session.

As will be understood, when operating to provide therapeutic light to a user, a light therapy application will illuminate (that is, cause the operating system of the computing system 10 to energize) the pixels of the various therapeutic areas 22. However, the light therapy application will do so while the computing system 10 (that is, the processor 12) is executing other applications which provide visual content via the windows 19, 20. In this manner, the user of the computing system 10 may perform other functions or tasks, such as reading, working, watching videos, playing a game, etc. while receiving therapeutic light from the areas 22. As used herein, therapeutic light generated by the light therapy application will typically have a wavelength in the range of 470 to 490 nanometers but could include other wavelengths of light outside of this range. Moreover, the preferred dosage of therapeutic light is typically at or near 5000 lux hours per day or more, but this dosage could be other amounts of therapeutic light, including in the range of 5000 to 10,000 lux hours per day, between 4000 and 5000 lux hours per day, between 4000 and 7500 lux hours per day, etc. The specific dosage may be dependent on the condition being treated, the severity of the condition, user or patient factors, etc.

FIG. 2 illustrates a more detailed block diagram of a computing system 30 which may be used to implement the integrated light therapy computing system 10 of FIG. 1 or any of the other computing environments described herein. In particular, the computing system 30 includes a microprocessor 32 coupled to a computer memory 34 to a computer screen or user display 36 and to one or more user input devices generally referred to as devices 38. The input devices 38 may be or may include, for example, a keyboard 38A, a mouse 38B, a microphone 38C and/or one or more other types of sensors 38D, that may provide user inputs to the computing system 30. Additionally, other input devices 39 may be connected to the processor 32 and provide other types of inputs thereto. The input devices 39 may include, for example, sensors that measure or sense environmental phenomena, movement, user actions, light, etc. The sensors 39 may be mounted on or adjacent to screen 36 or may be disposed at other locations with respect to the screen 36 and may sense or measure inputs related to the user as well as measurements or sensed phenomena regarding the environment in which the computing system 30 exists. The sensors 39 may include any types of sensors including microphones, video sensors, light sensors, cameras, motion detectors, etc. Still further, if desired, one or more external or peripheral light sources 40 (e.g., light bulbs, LED lights, etc.) may be disposed on or next to the screen 36 in a fixed or movable manner, such as on the top of the screen 36, at the bottom of the screen 36, on the sides of the screen 36, etc. The light sources 40 may be disposed at any other desired location and are electrically connected to and powered by or controlled by the microprocessor 32. Likewise, other output devices, such as one or more speakers and/or vibration devices 42 may be connected to and driven by the microprocessor 32.

As illustrated in FIG. 2, the memory 34 stores one or more applications 44 and an operating system 46 (which is implemented by the microprocessor 32). The operating system 46 may be any desired type or brand of operating system, such as a Microsoft Windows® operating system, an Apple® OS operating system, a Linux® operating system, etc. Still further, the applications 44 may include any desired types of applications including, for example, word processing applications, internet browsing applications, music applications, database storage and retrieval applications, control applications, gaming applications, communications applications such as internet telephony and video applications, etc. Of course one or more of the applications 44 may be implemented simultaneously on the microprocessor 32 using the operating system 46 in any typical manner. Importantly, one of the applications 44 may be or may include a light therapy application (generally designated as application 44A) which may be executed on the microprocessor 32 to implement and control one or more light therapy sessions using all or portions of the screen 36, as well as potentially one or more of the light sources 40 and/or other input and output devices 38, 39, 42. As will be understood, the light therapy application 44A may be executed on the microprocessor 32 alone, or in conjunction with (e.g., simultaneously with) one or more other applications 44.

Generally speaking, the light therapy application 44A may include a number of different components or routines that may be used to implement a light therapy session and to implement a light therapy regime comprising multiple light therapy sessions. In particular, the light therapy application 44A may include a configuration or set up routine that enables a user to program or set up the specifics of one or more light therapy sessions, such as the wavelength or wavelengths of light and the brightness or intensity of the light to use during a therapy session, where the light created during a therapy session will emanate (e.g., which portions of the computer screen 36 or light sources 40 will be used to create the therapeutic light for the light therapy session), the amount of light that needs to be received by the user during a light therapy session, whether the therapeutic light created during a light therapy session can change brightness or position on the screen 36, how to track the amount of light received by the user during a light therapy session, how often a light therapy session needs to occur (e.g., once per day, twice per day), etc.

Still further, the light therapy application 44A may include a session implementation routine that uses the configuration data provided by the configuration routine to implement a light therapy session using the computer system 30. In this case, the light therapy session implementation routine may illuminate one or more light sources (e.g., pixels of areas on the screen 36 and/or the light sources 40) to shine light towards the user using the computing system 30. The session implementation routine may use one or more input devices 38 and sensors 39 to detect the amount of light incident on the user's face, eyes, pupils, etc., may change the areas of the screen 36 or the number of the light sources 40, and/or may control the brightness of the lights on the areas of the screen 36 or the light sources 40 to provide a desired amount, brightness and wavelength of light to the user based on other actions that user is taking, such as based on the number and/or type of other applications 44 being implemented by the computing system 30, facial expressions being made by the user (e.g., squinting blinking, closing of eyes, etc. performed by the user), etc. Moreover, the session implementation routine may track the brightness of the light provided to the user during a session, may track the time that the user is exposed to the therapeutic light of the light therapy session, may track or calculate the lux (i.e., the lux hours) received by the user during a light therapy session, may stop a session when the user has received a desired amount of light or lux, and may store the specifics of a light therapy session after completion of a light therapy session, such as the amount of light or lux hours received by the user during the session, the time of the session, the duration of the session, and other parameters defining the session (such as the other applications being used by the user during the session, actions taken by the user, etc.) Of course, the processor (or light therapy application 44A) can specify the output of light detected or measured by sensors (or output by the various therapeutic light sources) as “lux hours,” wavelength intensity, etc. and the light therapy application 44A may or may not use sensors to detect the therapeutic light output. If external sensors 39 are used, these sensors may be connected to the processor 32 to provide feedback indicating when and if the proper dosage of light has been delivered so as to assure that the user is getting a total of, for example, 5000 lux hours throughout the day. Of course, the light therapy application 44A may add up the amount of time that the user's pupils are exposed to the therapeutic light and the therapeutic light may be delivered continuously or intermittently, such as 10 minutes at one time, 15 minutes at another, 5 minutes at another, etc. The light therapy application 44A may have an interface to show therapeutic light delivery progress throughout the day.

Still further, a therapy management routine may manage a therapy treatment by tracking the number of light therapy sessions provided to a user over time, providing light therapy session data or specifics for one more therapy sessions to a medical provider to assist the medical provider in tracking the effectiveness of the light therapy regime and to enable the medical provider or consultant to change the treatment being provided in the light therapy regime, such as the amount, brightness, lux, wavelength, etc. of the light to be received by the user during each session, increasing or decreasing the number of sessions, etc. The therapy management routine may allow the user to transmit the therapeutic information to a medical doctor's electronic medical record (EMR) system directly for the user as well as allow the user to provide anonymized information for research purposes to one or more researchers, to improve the device and outcome. The therapy management routine may also enable multiple users to use the same integrated light therapy device so that, for example, more than one family member can use it, and the management routine may track and report the light therapy data for each user separately. Likewise, the therapy management routine may remind the user of the need to start a session, send reminders to the user (via text messages or email for example), inform the user of the completion of a set of sessions, etc. The light therapy management routine may periodically provide the user with one more tests which the user may take to enable the user or a medical provider to assess the need for, the value of or the effectiveness of the light therapy treatment being provided by the application 44A. Such tests could include measurements of depression rating scales, such as the Hamilton Depression Rating Scale, the Beck Depression Inventory, Center for Epidemiologic Studies Depression Scale, EQ-5D, the Montgomery-Åsberg Depression Rating Scale, the Social Problem-Solving Inventory-Revised Scale or any other depression rating scale. The tests could also or instead measure anxiety based on any number of anxiety rating scales, including the GAD 7 scale, the State-Trait Anxiety Inventory (STAI), the Beck Anxiety Inventory (BAI), the Hospital Anxiety and Depression Scale-Anxiety (HADS-A) or any other anxiety rating scale. Still further, the tests could measure sleep quality using any sleep rating scale such as the Pittsburgh Sleep Quality Index (PSQI), the Parkinson Disease Sleep Scale (PDSS), the Parkinson Disease Sleep Scale-2, SCOPA-Sleep (NS Subscale), or any other sleep rating scale. Any other rating scales, measures or progress trackers can also be used based on the current condition being treated.

More particularly, during operation, one or more of the applications 44, including the light therapy application 44A, may be executed simultaneously by the microprocessor 32 using any desired or available operating system 46. The applications 44 may use the input devices 38, including a keyboard, a mouse, microphones and other sensors 39 to provide content to and to control one or more visual displays (windows) on the screen 36 with respect to the applications 44. In addition, the light therapy application 44A may operate to illuminate various pixels or lights on the screen 36 and/or one or more of the light sources 40, to provide a light therapy session to the user while the user is viewing the display screen 36, including viewing other windows or visual content provided on the display screen 36 by one or more other applications 44 which are not the light therapy application 44A.

In one embodiment, the light therapy application 44A may be executed or initiated by the user via the input devices 38 and the application 44A may operate to provide a fixed or variable amount of light via a set of pixels on the screen 36 directed towards the user's eyes. The pixels or areas of the screen 36 used to provide the light for the light therapy session may be fixed or may change during the light therapy session. For example, the light therapy application 44A may illuminate or energize a fixed band or other area of pixels on the screen 36, such as at the top of the screen 36, at the bottom of the screen 36, on the sides of the screen 36 and/or at any other locations on the screen 36. In this case, other applications 44 executing on the microprocessor 32 may use other portions of the screen 36 (e.g., the center portions of the screen 36) to provide their other non-therapeutic content to the user. In other cases, the light therapy application 44A may illuminate pixels on the screen 36 that are not being used by the other applications 44 and thus the amount of light or the areas of the screen 36 used by the light therapy application 44A at any point in time may change and may be dependent on the other applications that are executing on the microprocessor 32 and the visual content that these other non-therapeutic applications are providing. In this case, the user may make changes to the size or positions of the windows associated with the other, non-therapeutic applications which will change the locations and amount of light or the amount of the display screen 36 being illuminated or controlled by the light therapy application 44A. In still other cases, the light therapy application 44A may enable a user to select, specify and/or change the portions of the screen 36 being used for the light therapy session at any particular time or may enable the user to specify the portions of the screen 36 to be used for providing therapeutic light at the beginning of the light therapy session. Thus, in one case, the operating system 46 may make the areas of the screen 36 used by the light therapy application 44A dependent on or subject to the other applications 44 being executed on the microprocessor 32, so that the light therapy application 44A must use spaces on the screen 36 not currently being used by the other applications. In this case, the light therapy application 44A may increase (or decrease) the light provided to the user during the light therapy session by using one or more of the light sources 40 and/or by changing the brightness or intensity of the pixels on the screen 36 used to provide therapeutic light. In other cases, the operating system 46 may require the other applications 44 executing on the microprocessor 32 to use only the areas of the screen 36 not reserved for or used by the light therapy application 44A. In still other embodiments, the light therapy application 44A may use one or more of the light sources 40 to provide all of the therapeutic light or to provide additional therapeutic light to the user during a light therapy session. Thus, if desired, the light therapy application 44A may simultaneously use both the light sources 40 and one or more areas or portions of the screen 36 to provide therapeutic light to the user for a light therapy session or may switch between the light sources 40 and one or more areas of the screen 36 during a light therapy session. Still further, the light therapy application 44A may enable a user to, or may automatically select or change the areas of the screen 36 to use, one or more of the light sources 40 to use, the brightness or intensity of the light provided by the light sources 40 and the illuminated areas of the screen 36, the wavelengths of the light provided by these light sources, or any combination thereof to control the amount of light that is incident on the user, and thus the amount of or the rate of therapeutic light that user receives during any particular light therapy session.

Still further, the light therapy application 44A may track and record one or more parameters associated with a light therapy session including ongoing or changing values of particular parameters of the provided therapeutic light during a light therapy session. For example, the light therapy application 44A may track and record the amount of therapeutic light (associated with the light therapy session) that is provided to the user at any particular time, including, for example, the number of the light sources 40 being used, the intensity or brightness of the light sources 40 and the wavelength(s) of the light provided by each of the light sources 40 at any particular time, the number and location of pixels on the screen 36, the intensity or brightness of the pixels on the screen 36 and the wavelength(s) of the light provided by each of the pixels on the screen 36 at any particular time when these pixels are being used for a light therapy session. The light therapy application 44A may estimate, detect (using one or more sensors) or may otherwise record the distance of the user from the screen 36 to determine the amount of therapeutic light that is reaching the user's retina based on the position, amount and type of light sources used for providing the therapeutic light, the brightness of the light sources, and the wavelength of therapeutic light directed towards the user by the either or both the pixels on the screen 36 and the light sources 40. The light therapy application 44A may integrate, add or otherwise determine the amount of light incident on the user's face, eyes, pupils, retina, etc. over time during a light therapy session to determine the amount of lux (lux hours) being received by the user during the light therapy session. The light therapy application 44A may execute a light therapy session for a predetermined amount of time or may end a light therapy session when the desired amount of light or lux hours has/have been provided to the user, as determined by the light therapy application 44A.

Still further, in other embodiments, the light therapy application 44A may use one or more sensors, such as any of the sensors 39, to detect the amount of therapeutic light being directed onto or that is incident onto a user's face, the brightness of the therapeutic light incident on a user's face, etc. Additionally, the application 44A may use sensors 39 and any processing routine needed to detect the movement of the user's eyes and/or eyelids, and the position of user's eyes and/or eyelids, to detect whether the user is looking at the screen 36 or has his or her eye(s) closed or open, to determine the amount of time and the brightness of the light that is actually incident on the user's face, eyes, retina or cornea, etc. during a light session therapy session, etc. The light therapy application 44A may use the sensors 39 to detect or determine the amount of therapeutic light incident on the user's face, eyes, retina, etc. at any particular time and to accumulate, add or integrate the amount of therapeutic light that has actually been received by the user during a light therapy session to determine whether the user is receiving the appropriate amount of therapeutic light or to determine the actual amount of therapeutic light provided to the user during a particular light therapy session. In some cases, the light therapy application 44A may change the amount of light (by increasing or decreasing the amount or brightness or wavelength of therapeutic light provided by the screen 36 and/or the light sources 40) based on the use of other applications 44, which may operate to use more or less of the screen 36. When the light therapy application 44A determines that a light therapy session is over, the application 44A may inform the user of the end of the session, or may automatically shut off or release control of the light sources 40 and any of the pixels on the display 36 associated with or being used by the light therapy application 44A to provide a light therapy session. The light therapy application 44A may thus operate during a light therapy session to change the number and location of the light sources (both the light sources 40 and pixels of the display 36), the position of the light sources, the brightness or intensity of the light sources, the wavelength(s) of light emitted by the light sources, etc. during a light therapy session and may determine, based on one or both of the lights and lighting parameters (light intensity, wavelengths, position of the light sources, etc.) being used at any particular time in the light therapy session and feedback by sensors 39 regarding the amount (and other parameters) of light reaching the user's eyes to determine the amount of therapeutic light being provided to the user during a light therapy session. Of course, the lighting parameters (e.g., position, brightness, intensity, wavelengths, etc.) of the light provided by the light therapy application 44 may change from time to time (e.g., second to second, minute to minute, etc.) based on activities the user is taking on the screen 36 with respect to other applications 44 executing on the computer system 30. Still further, the light therapy application 44A may track (and store in the memory 34) the amount of light (i.e., lux) received by the user during each light therapy session and the time and days of each light therapy session. The light therapy application 44A may additionally track sessions over time so as to keep track of the amount of light therapy that the user is being provided over time.

In still other cases, one or more of the sensors 39 may include or be medical sensors, such as blood pressure sensors (of any suitable kind), pulse sensors, blood oxygen sensors, heart rate sensors, etc. In one case, the sensors 39 may include one or more retinal scanners, such as a fundus camera, or other optical imaging system that is mounted on, integrated with or otherwise attached to the system that provides the user display (e.g., a computer screen, AR googles, a VR headset, etc.). In one such system, a retinal camera may be clipped onto an AR headset as an add on device. Such a retinal scanner may be aligned with the axis of the user's eye or pupil and provide controlled light or illumination to the retina to image the retina. In particular, the optical imaging system may be precisely aligned with the optical axis of the eye and provided controlled illumination, typically near-infrared for alignment and white light for imaging, within regulated safety limits to perform optical imaging and detection. In these cases, the light therapy application 44A may use feedback from the medical sensors to change or alter the amount, type, brightness or other parameter of the therapeutic light (or other operations being performed) to provide a safer or more effective environment for the therapy session to determine the effectiveness of the therapy or other medical conditions of the user before, during or after a light therapy session. In addition, the therapy application 44A may store the captured medical information for analysis later or for sending to a medical professional. This data may also be used to determine the best settings and configurations for providing therapeutic light, and/or for determining the effectiveness of one or more therapeutic sessions.

As noted above, the light therapy application 44A may provide light used during each light therapy session via one or more of the pixels on the screen 36 and the light sources 40, and may use feedback regarding the amount of light being provided to and/or absorbed by the user (as detected by one or more sensors 39) to determine the amount of light (lux) provided to a user during a light therapy session. The user feedback may include detecting eye movement and position (e.g., where the user's eyes are looking at any particular time), whether the user's eyes are open or closed and for how long, the size of the user's pupils, etc. to determine the amount of therapeutic light being received, the brightness or intensity of the light incident on the user's eyes, etc. The sensors 39 may also detect user motions in order to control the amount of light provided to the user, such as by increasing or decreasing the amount or intensity of therapeutic light provided by the screen 36 or the light sources 40, and changing the wavelength of light provided by the screen 36 or the light sources 40, etc. based on, for example, user facial expressions or actions (e.g., squinting of the eyes).

In still other cases, the light therapy application 44A may be integrated into, or may be made to be a part of or run in conjunction with other applications 44. As a result, the amount of light and/or the areas of the screen 36 used for providing light therapy may change during a light therapy session based on the operation of the other applications 44. Of course, if desired, the light therapy application 44A may be an application that runs in the background of the computing system 30. In still other cases, the light therapy application 44A may be an active application that provides the therapeutic light for a light therapy session as part of another application, such as a meditation application or an application designed to provide the user with entertainment. In this case, the screen display provided by the meditation or entertainment application may provide a changing visual display that is pleasing, soothing or entertaining to the user, but that includes therapeutic light needed for a light therapy session. For example, a meditation application may provide a visual scene on the screen 36 that changes over time, but that includes bright spots or lighting associated with or needed for a light therapy session. Such bright spots may be depicted as one or more suns in a landscape or other intense lights as part of a visual scene (e.g., headlights of a car, bright reflections on the surface of a lake, etc.) In this case, the bright portions of the scene may provide therapeutic light of sufficient brightness and wavelength and duration to the user to be useful for a light therapy session, while other areas of the screen (or the visual scene), do not provide the light of the appropriate wavelength or brightness for a light therapy session. The advantage of such applications is that they provide the user with an added benefit, such as meditation or entertainment, while also providing light therapy by incorporating scenes that include light sufficient for a light therapy application. Of course, these meditation or entertainment applications may include or provide other sensory information, such as sounds, or vibrations or smell and/or other haptic feedback, etc., in conjunction with the visual information.

Still further, the light therapy application 44A may operate to interleave or intersperse the pixels used on the display screen to provide therapeutic light with the pixels of the display screen used by other applications 44. For example, the light therapy application 44A may detect unused pixels (or other areas of the screen), i.e., those not being used by other, non-therapeutic applications 44, and may use those pixels for providing therapeutic light. These pixels may be outside of any of the windows used by the other active non-therapeutic applications 44 or may be pixels within the windows used by other active non-therapeutic applications 44. In some cases, the light therapy application 44A may replace or use pixels used by other non-therapeutic applications 44 with therapeutic light. In this case, for example, the light therapy application 44A may determine that one or more pixels being used by a non-therapeutic application 44 may be replaced or overwritten so as to provide therapeutic light. As an example, if a pixel being illuminated by a non-therapeutic application 44 falls within a particular range of color and/or brightness (but is still providing non-therapeutic light), the light therapy application 44 may alter that pixel to provide therapeutic light. In other cases, the light therapy application 44A may simply change various pixels used by a non-therapeutic application 44 to illuminate in a manner that provides therapeutic light and may do this in a random pattern or any desired non-random pattern, such as every fifth pixel.

In still another embodiment, the light therapy application 44 may observe or detect the particular type (color or wavelength and intensity or brightness) of the light being provided by each of the pixels in a display screen 36 being controlled by a non-therapeutic application 44 and may determine when (and for how long) each pixel is providing therapeutic light as a happenstance of the operation of the one or more non-therapeutic applications 44. The light therapy application 44A may determine whether each pixel is providing therapeutic light or not based on the control signal provided to that pixel by the operating system and or the graphics controller (card). The light therapy application 44A may then simply sum up this therapeutic light over time to determine if (and when) the user is receiving a sufficient dosage of therapeutic light as a result of the operation of the non-therapeutic applications 44. The light therapy application 44A may, if desired, augment the therapeutic light from the non-therapeutic applications by illuminating pixels of the screen 36 or peripheral light sources 40 with therapeutic light as described above, but in some cases this may not be necessary. In another case, the light therapy application 44A may use one or more of the sensors 39 to measure the amount of therapeutic light being provided to a user by non-therapeutic applications 44 over time and/or by a combination of therapeutic light being provided by non-therapeutic applications 44 and by the light therapy application 44A to determine when a user has received a proper or sufficient dosage of therapeutic light. Thus, in some cases, the light therapy application 44A may simply measure and track the amount of therapeutic light being provided to the user by other non-therapeutic applications 44 and determine when and if the user has received a desired dosage of therapeutic light as a result of the use of non-therapeutic applications 44. The light therapy application 44A may also augment the therapeutic light provided by non-therapeutic applications 44 to assure that the user receives a proper or desired dosage of therapeutic light while the user is viewing displays associated with non-therapeutic applications 44.

The screen 36 of FIG. 2 may use any digital technology that provides sufficient light for a light therapy session, and may be, for example, a liquid crystal display (LCD), a cathode ray tube (CRT), a plasma screen display, or any type of light emitting diode (LED) display, such as an organic light emitting diode (OLED) display. However, in at least one preferred embodiment, the screen 36 may include or be made of mini-LED or micro-LED components, which are relatively new in the art of display technology. Generally speaking, mini-LEDs and micro-LEDs are able to be controlled to provide more or brighter light per pixel than other types of LED display technology, and thus are more suitable to provide light therapy via a computer screen than other types of pixel based screen or display technology. Additionally, the light sources 40 may be any desired type of lights, such as LEDs or incandescent lights, or other types of lights (including mini- or micro-LEDs) that provide the appropriate wavelength and the brightness required for a light therapy session. Still further, the computing system 30 of FIG. 2 may be implemented as any type of computing device or system, such as a desktop computing system, a laptop, a phone, an e-reader, a smart TV, etc. and, in one or more preferred embodiments, may be any type of extended Reality (XR) computing system including any Augmented Reality (AR) computing system, Mixed Reality (MR) computing system, Virtual Reality (VR) computing system, spatial computing device, etc. Moreover, while the computing system 30 of FIG. 2 is illustrated as including a single desktop type interface screen or display 36, the computing system 30 of FIG. 2 could including any number of user interface displays (e.g., two, three, four, etc.) disposed and configured to operate together in any desired manner (e.g., via the operating system 46 of the computing system 30). Moreover, the displays 36 of the computing system 30 may include a separate display for each eye of a user (which is typically the case in XR devices) or may include one or more displays that emit light that reaches both eyes of a user, or may include a single display that emits light that reaches only one eye of the user.

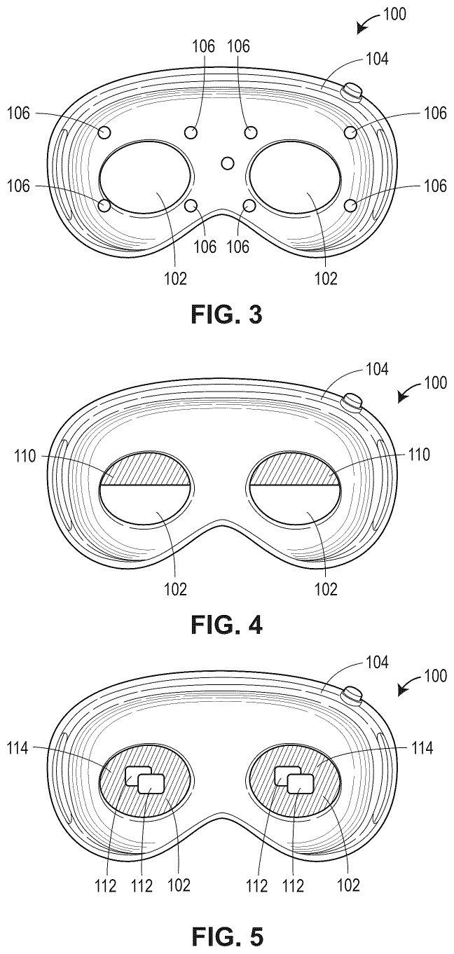

FIG. 3 illustrates an example of a computer system such as that of FIG. 2, incorporated in or comprising a set of extended Reality (XR) goggles or glasses 100, such as VR, AR, MR, XR, spatial computing, etc. goggles 100 (generally referred to herein as VR goggles) that may be used to implement the integrated light therapy techniques described herein. The VR goggles 100 may be, for example, Google Glass®, Meta Quest®, Apple Vision Pro® headsets, or any other type or kind of virtual reality glasses or goggles that includes one or more screens or displays. The Apple Vision Pro® headsets are especially useful in this context as they incorporate or have screens that include micro-LED display technology. In any event, as illustrated in FIG. 3, a separate display screen 102 or eyepiece 102 for each eye is mounted in headgear or other support structure 104 so that the display screens or eyepieces 102 are disposed very close to a user's eyes. The support structure or head piece 104 may also include various sensors 106 disposed therein, and these sensors may include light or other ambient condition detection sensors, video cameras, eye tracking sensors, motion sensors, and/or any other desired types of sensors described above as, for example, sensors 18 and 39 of FIGS. 1 and 2. The sensors 106 may, for example, track or measure elements of the environment and/or may detect or track a user's facial and/or eye movements (such as the position of an eyelid, whether an eyelid is open or closed, where an eye is looking, the size of the pupil or the retina exposed to light, etc.) While not shown in FIG. 3, the set of VR goggles 100 includes one or more microprocessors and one or more memories, and may include other input devices and peripheral devices, such as any of those described with the respect to FIG. 1 and and/or available with any commercially available VR googles.

As will be understood, the VR goggles 100 may implement a light therapy session by providing light to various parts of the user's eyes, via the displays or screens in the eyepieces 102, in any of the various manners described herein, to thereby provide therapeutic light to a user in addition to providing other visual content or windows to the user to perform other tasks, such as other tasks generally performed on a computer or a set of VR goggles. In some cases, such as with the Apple Vision Pro headset, the VR goggles 100 may include one or more video cameras or other sensors (e.g., microphones) mounted thereon to view out of the front and/or sides of the headset 104 to detect the ambient environment. The outputs of these sensors may be used in part or in whole to provide video and sound to the user via the screens 36 and speakers disposed within the headset 104. As will be understood, a light therapy application may be executed on the processor of the VR goggles 100 to provide light therapy via the screens of the eye pieces 102, while other applications executed on the microprocessor of the VR goggles 100 enable the user to view the ambient environment, view and use other computer generated windows, perform searches or other internet communications, use one or more word processing or other proprietary or non-proprietary applications, etc.

Of course, the light therapy application executed on the VR goggles 100 of FIG. 3 may use any parts of the screens associated with the eye pieces 102 to provide therapeutic light for the light therapy session to a user. As an example, FIG. 4 illustrates the VR goggles 100 in which the light therapy application (44A of FIG. 2) provides therapeutic light (having a desired intensity and wavelength) via a strip of pixels 110 on the screen of the eye piece 102. In this example, the strip of pixels 110 is disposed at and across the top portion of each of the screens of the eye pieces 102 and is created by energizing or controlling the micro-LEDs within the strip of pixels 110 of each of the eye pieces 102 to illuminate or create the therapeutic light.

In a similar manner, FIG. 5 illustrates an example set of VR goggles 100 in which the light therapy application may cause the therapeutic light to emanate from various unused portions of the screens in each of the eye pieces 102. Thus, as illustrated in FIG. 5, the user can view one or more windows 112 providing visual content from other, non-therapeutic applications near the center of the screens of the eye pieces 102, while the light therapy application provides therapeutic light from the areas 114 of the screens of the eye pieces 102 that are not being used by the other applications. While, in this example, the therapeutic light is provided from areas 114 of the screens of the eye pieces 102 at the outer portions of the screens of the eye pieces 102, other areas could be used as well or instead. Additionally, and as noted above, the therapeutic light can be provided from fixed or moving areas 114 of the screens of the eye pieces 102 and the visual content provided by the other applications executing on the VR goggles 100 may be sized or manipulated to avoid those fixed or moving areas 114. In other cases, the areas 114 of the screens of the eye pieces 102 that are used for providing therapeutic light may be managed or changed so as to not interfere with the visual content or windows associated with the other non-therapeutic applications executing on the VR goggles. In still other cases, the areas or portions 114 of the screen used for therapeutic light may overlap and be intermingled with the areas or portions 112 of the screen used by non-therapeutic applications. Likewise, the light therapy application may enable a user to select the sections 114 of the screens of the eye pieces 102 to use to provide therapeutic light. Of course, as noted above, the other non-therapeutic applications may be any types of applications, such as video applications, word or data processing applications, gaming applications, communication applications, etc.

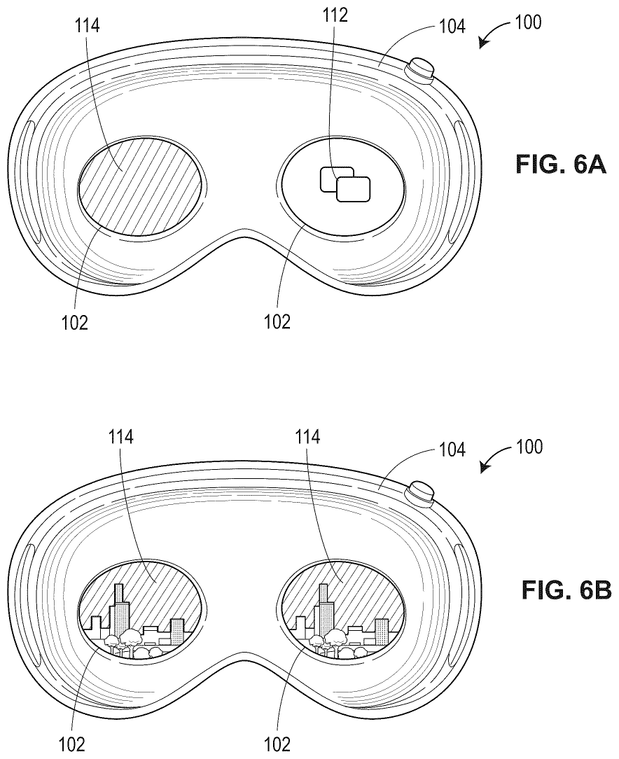

As another example, FIG. 6A is a depiction of a third embodiment of a light therapy screen display implemented using a set of virtual reality (VR) or (XR) goggles 100 in which the light therapy application provides therapeutic light via only one of the displays of the eyepieces 102 at a time. That is, in this example, one of the displays of the eyepieces 102 (e.g., the left display of FIG. 6A) may be used to provide therapeutic light, while the other one of the displays of the eyepieces 102 (e.g., the right display of FIG. 6A) may be used for other computing activities, such as other applications. The light therapy application may, if desired, use only one display for an entire therapy session or may switch between the use of the left and right display throughout a session, for example, in a periodic or non-periodic manner, to reduce eye fatigue. Of course, the therapeutic light may be provided using the entire screen display of one of the eyepieces 102 or may only use part of the screen display of one of the eyepieces 102 in these examples.

FIG. 6B illustrates another example of an integrated light therapy system implemented using a set of virtual reality (VR) or (XR) goggles 100 in which the light therapy application provides therapeutic light intermingled with visual content provided by one or more other applications or in which the light therapy application is integrated with another non-therapeutic application to provide visual content that includes some therapeutic light intermingled with other (non-therapeutic) visual content. In the example of FIG. 6B, an integrated therapy and non-therapy application may provide a visual scene comprising an outdoor scene, in this case a cityscape via the screens on the eye pieces 102. Here, however, the sky 114 of the cityscape may be altered to provide therapeutic light that looks like a brighter (or different color) sky than might otherwise be displayed so that the light from sky of the scene conforms to and provides the therapeutic effect. In this example, the therapeutic light 114 (in the form of an altered sky) may be provided in a virtual reality environment in which the scene is a completely virtual scene, or may be provided in a mixed reality environment in which at least some portions of the scene are based on an actual or real-life environment, such as that provided by a video camera providing input of an actual real-life scene in which the user is present. In this manner, a user may be able to receive therapeutic light by receiving an altered version of the environment in which the user exists, such that the altered version includes therapeutic light substituted for some portion of the actual environment. Of course, FIG. 6B illustrates only one example of a manner of integrating therapeutic light with other visual content. If desired, the operating system of the computing system may enable the user to select or provide this integration, in the form of an accessibility setting enforced by the operating system. Thus, an accessibility section of the operating system may include controls to enable a user to specify the use of an integrated light therapy application with a non-therapeutic application or with the manner in which an integrated light therapy application may interact, via the operating system, with other non-therapeutic applications.

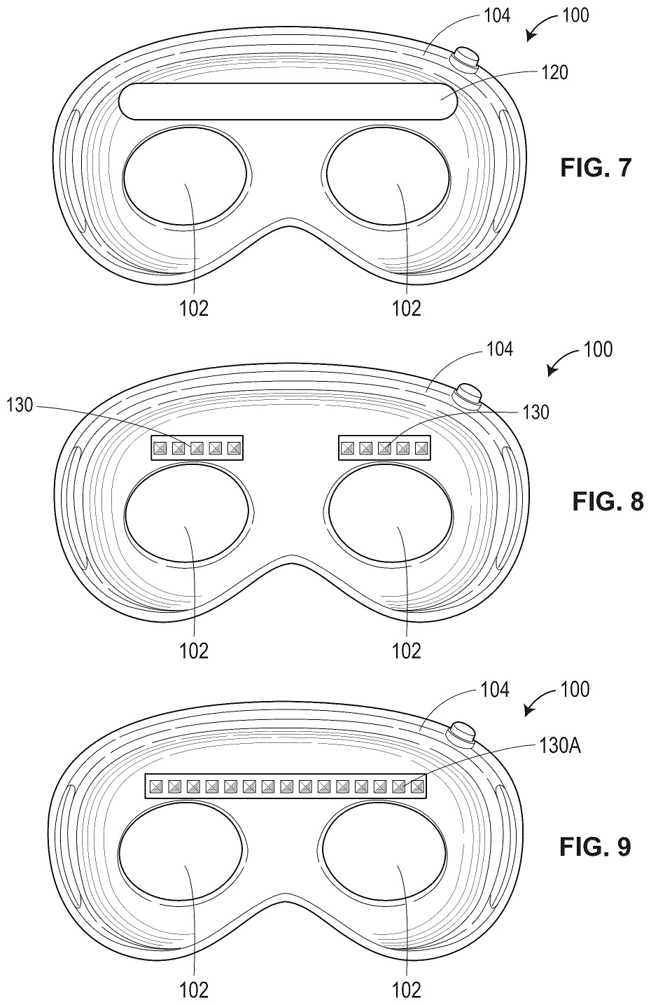

FIG. 7 illustrates a further example of a manner of providing light therapy via a set of VR goggles 100. In this case, a set or strip of lights 120, which may be micro-LEDs, is installed or mounted in the headpiece 104 of the VR goggles 100, for example, above the displays of the eye pieces 102. The lights 120 (which may correspond to the light sources 40 of FIG. 2) may be illuminated or controlled independently of the displays on the eye pieces 102 by the light therapy application executing on the VR goggles 100 to provide therapeutic light from the strip of lights 120 while the user is viewing other content on or via the displays of the eye pieces 102. In this case, the light therapy application may use only the lights in the strip of lights 120 for providing therapeutic light, or may use both lights in the strip of light 120 and one or more areas of pixels on the screens of the eye pieces 102 to provide therapeutic light.

FIGS. 8 and 9 depict other manners in which additional or peripheral light sources can be installed in a set of VR goggles 100 to provide therapeutic light for one or more light therapy sessions. FIG. 8 illustrates a set of VR goggles 100 having a separate set of LEDs 130 or other types of lights disposed above each of the eye pieces 102 and, similar to the micro-LED strip of FIG. 7, these lights 130 may be illuminated or controlled separately by the light therapy application to provide light therapy to a user or wearer of the goggles 100 while the user is viewing other content via that eye pieces 102. FIG. 9 illustrates a similar embodiment to that of FIG. 8 in which a single strip or set of LED lights 130A (or other types of lights) are disposed in the VR goggles 100 (i.e., in the support 104 of the goggles 100) to provide therapeutic light to the user while user is viewing other visual information provided on the displays of the eye pieces 102 by other applications. Moreover, each of these light sources can be controlled by the same CPU which can then provide all the functionality described above.

Of course, as will be understood, any of embodiments of FIGS. 3-9 may use one or more sensors disposed on or in the VR goggles 100 (such as sensors embedded into the support piece 104) to detect the amount, wavelength, and/or brightness or intensity of light that has been emitted by the display based or peripheral light sources, to detect the position and other aspects of the user' eyes (such as whether the user's eyes are open or closed, how wide open the user's eyes are at any point in time, pupil size, the direction in which the user is looking, etc.), as well as to detect and measure the amount or brightness of the light incident on the user's face, eye, pupil, etc. The light therapy application may use the outputs of these sensors in any desired manner to determine how much therapeutic light the user is receiving or absorbing during a particular light therapy session, or at any particular time during a light therapy session. The light therapy application may then integrate or add the determined amounts of light to measure or track the amount of lux (lux hours) that is received by the user, and to determine whether that amount of lux hours is sufficient for the completion of a light therapy session that is currently being performed.

Likewise, as described above, the light provided by a light therapy application on any of the VR goggles of FIGS. 3-9 may be altered or changed during a light therapy session, such as by changing the amount or brightness of the light sources that are illuminated by the light therapy application, by changing the positions of the LEDs on the screens or displays of the eye pieces 102 that are used to provide therapeutic light associated with a light therapy session, by changing the brightness and/or wavelength of the therapeutic light that is provided by the displays of the eye pieces 102 and/or the sperate light sources 120, 130, 130A, etc. In one example, the light therapy application may detect if too much or too little therapeutic light is incident upon the user's eyes based on the other windows that user is viewing and/or based on other actions by the user (such as if the user is squinting his or her eyes), and may decrease or increase the amount of therapeutic light being provided during the light therapy session in order to enable the user to open their eyes more and absorb more of the therapeutic light or to make the light associated with the light therapy session be less noticeable or distracting to the user with respect to the other activities the user may be performing. The light therapy application may alter the amount of light by changing the size or areas of the screens of the eye pieces 102 used to provide therapeutic light, by increasing or decreasing the number of peripheral lights 120, 130 and 130A used to provide therapeutic light, by increasing or decreasing the brightness and/or wavelength of light emitted by the light sources or pixels of the screens of the eye pieces 102 emitting therapeutic light, etc.

It will be understood, of course, that the configuration and location of the light sources, including the pixels used on the displays of the eye pieces 102 as well as the LED or other lights 120, 130, 130A separately mounted into the headpiece 104 of the VR goggles 100 are exemplary only and that other numbers and positioning of peripheral lights and/or the areas of the displays of the eye pieces 102 used by the light therapy application may be changed from those illustrated and described herein.

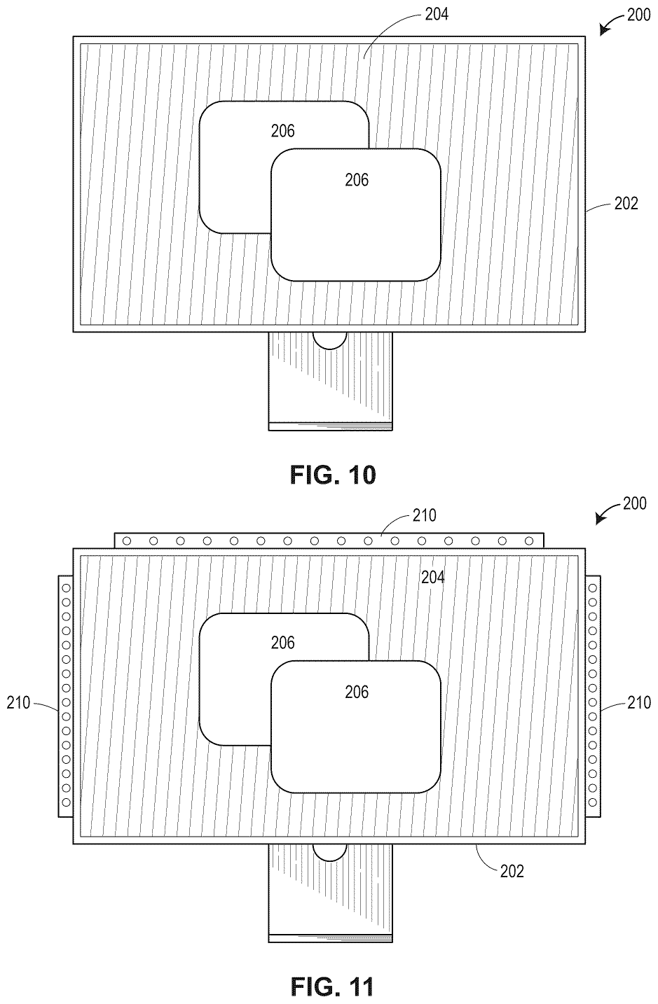

FIGS. 10 and 11 illustrate example configurations of a display alone (FIG. 10) and a display with a set of peripheral light sources (FIG. 11) for an integrated light therapy system implemented using a typical desktop computer 200 having a desktop screen or display 202. In the embodiment illustrated in FIG. 10, a light therapy application may provide therapeutic light via a section 204 of the screen 202 which may surround or be on one side or another side of windows or displays 206 associated with or created by different (non-therapeutic) applications executing on the computer 200. Of course, while the therapeutic light screen sections 204 are illustrated as surrounding the windows 206 used or created by other applications, the screen sections 204 may use up more less area of the entire screen 202, may move around the screen 202, may be located on one side of the other side of the screen 202 or may take on any other desired configuration using any other portions of the screen 202.

The desktop embodiment illustrated in FIG. 11 is similar to that of FIG. 10, except that it includes one or more separate sets of peripheral lights 210, each having one or more light sources therein, disposed on or adjacent to the screen 202. Here, the additional light sources 210 are depicted as being mounted on top of and on the sides of the screen 202, but these light sources 210 could be mounted in other locations and in other manners as well (and do not need to be mounted on the screen 202 at all). As noted above, these additional light sources 210 may be controlled by a light therapy application executing on the computer system 200 in conjunction with one or more displays areas 204 on the screen 202 to provide therapeutic light, and these peripherals light sources 210 may be used alone, or in conjunction with one or more portions 204 of the screen 202 to provide therapeutic light. Of course the peripheral light sources 210 may be any desired types of light sources suitable for providing light as part of a light therapy session, including for example, LED lights, micro-LED lights, incandescent lights, etc., which provide enough light at the wavelength needed to provide therapeutic light. Likewise, as noted above, the peripheral light sources 210 may be connected to the computer 200 and controlled by the light therapy application executing on the computer 200. Still further, the peripheral light sources 210 may be mounted on or in bevels or stands the enable the user or the computer (or light therapy application 44A) via an attached motor (not shown), to the change or adjust the positioning of the light sources 210 and/or to change the angle at which light from the peripheral light sources 210 is directed at the user.

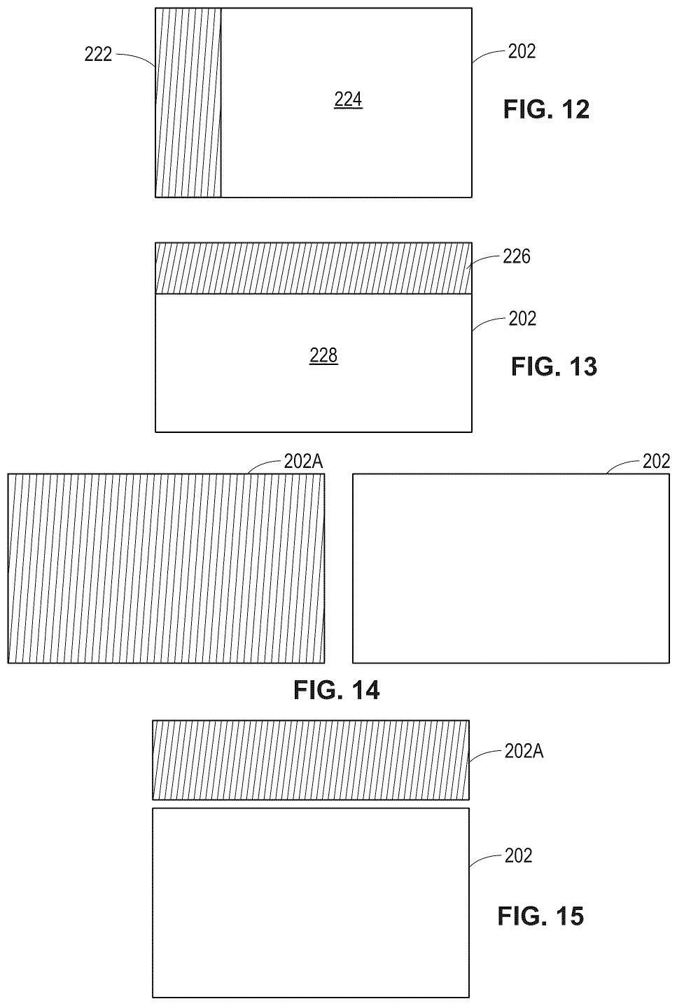

FIGS. 12-15 illustrate other example desktop computer screen set ups and

configurations which could be used to provide therapeutic light in addition to other content. For example, FIG. 12 illustrates a single desktop computer screen 202 in which the light therapy application creates a window of therapeutic light 222 on the left side of the screen and the right side of the screen 224 is used by other applications. The size and location of the therapeutic light window 222 may be set or enforced as a fixed window using the operating system setup or configuration or may be movable by the light therapy application 44A and/or a user.

FIG. 13 illustrates another example of single desktop computer screen 202 in which the light therapy application creates a window of therapeutic light 226 on the upper portion of display screen 202 and the lower portion 228 of the display screen 202 is used by other applications. The size and location of the therapeutic light window 226 may be set or enforced as a fixed window using the operating system setup or configuration or may be movable by the light therapy application 44A and/or a user.

FIG. 14 illustrates an example of a light therapy application set up that uses a desktop computer system that includes multiple separate screens 202 and 202A, in this case placed side by side. In this example, the light therapy application provides therapeutic light via one of the screens (e.g., the left screen 202A) while other applications executed on the computer system use the other screen (the right screen 202). In a similar manner, FIG. 15 illustrates an example of a light therapy application set up that uses a desktop computer system that includes multiple separate screens 202 and 202A in this case placed one above the other. In this example, the light therapy application provides therapeutic light via one of the screens (e.g., the upper screen 202A) while other applications executed on the computer system use the other screen (the lower screen 202). Of course, the light therapy application may use all or only a portion of one of the screens (202A) to provide therapeutic light during a light therapy session, and the positioning of the screens or the therapeutic light on the screens 202 and 202A may be the same or may change during a therapy session, from session to session, or in any other manner.

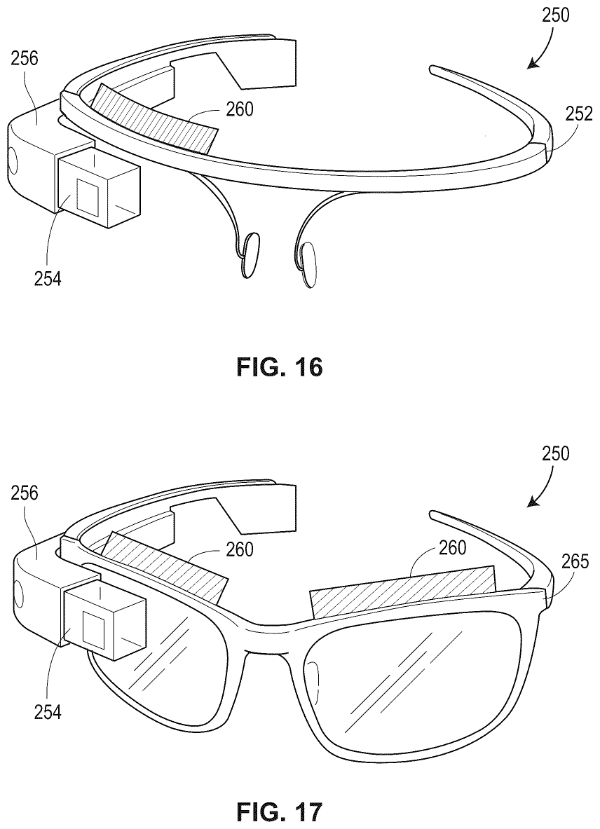

FIGS. 16 and 17 illustrate an example integrated light therapy device configured to be implemented as a set of MR glasses 250, such as Google Glass®. In the example of FIG. 16, a set of VR glasses 250 may a support frame 252, an eye piece 254 and a computer 256 mounted on the frame 252, and may include a light source (not shown) attached to or integrated with the housing of the computer 256 to provide therapeutic light to the user's eye in addition to other computer generated content. Alternatively or additionally, a separate light source 260 may be mounted near the eye piece 254 on, for example, the frame 252 and be attached or controlled by the computer 256 to provide therapeutic light to the user's eye. As with any of the embodiments discussed herein, the peripheral light sources (the light source 260 of FIG. 16) may be connected to and driven by the battery pack of the computer or may include a separate battery, not shown, which provides power to the peripheral light source. In the example of FIG. 16, the light source 260 may be implemented as any light source that provides sufficient light or lux hours at the appropriate wavelengths to the user's eye, including, for example, LEDs, mini-LEDs, micro-LEDs, fluorescent, incandescent, etc. lights.

FIG. 17 illustrates an embodiment of a set of MR glasses 250 having therapeutic light sources, such as LEDs or strips of micro-LEDs 260, mounted on a frame 265 to provide therapeutic light to both eyes of the user. Of course, the light sources 260 may be mounted above or at the top of the eye, or may be mounted on the side or below the eye in any desired manner and of course may be connected to and controlled by the computer 256 of the MR glasses 250 to provide therapeutic light while the user is viewing other computer content via the eyepiece 254 and/or while the user is viewing the real world environment through the eye piece 254. This configuration enables the user to obtain therapeutic light while interacting with the real world environment and/or interacting with other computer applications via a display on the eye piece 254. Of course, the therapeutic light sources may be provided to both eyes or only one eye, such as on the eye that does not see the eye piece 254 driven by the computer 256.

While the example computer systems depicted and described herein with respect to FIGS. 1-17 include three specific forms of a computer system and a computer screen (e.g., a desktop computer with a desktop screen, a set of VR goggles and a set of MR glasses) that could be used to implement the integrated light therapy system described herein, other forms of computer systems and computer screens could be used instead, including laptop computers, mobile phones, wall mounted or standalone smart televisions, etc. Moreover, these other computer systems and any of the systems described herein may be used with or without peripheral light sources. The use of peripheral light sources may not be necessary, especially when the screens of the computer devices are able to produce the necessary lux hours for therapeutic light, meaning when the screens of these devices can produce the wavelengths and light intensity necessary to provide a therapeutic dosage of light. Generally, the use of screens that use mini-LED or micro-LED technology is considered preferable to obtain this advantage.

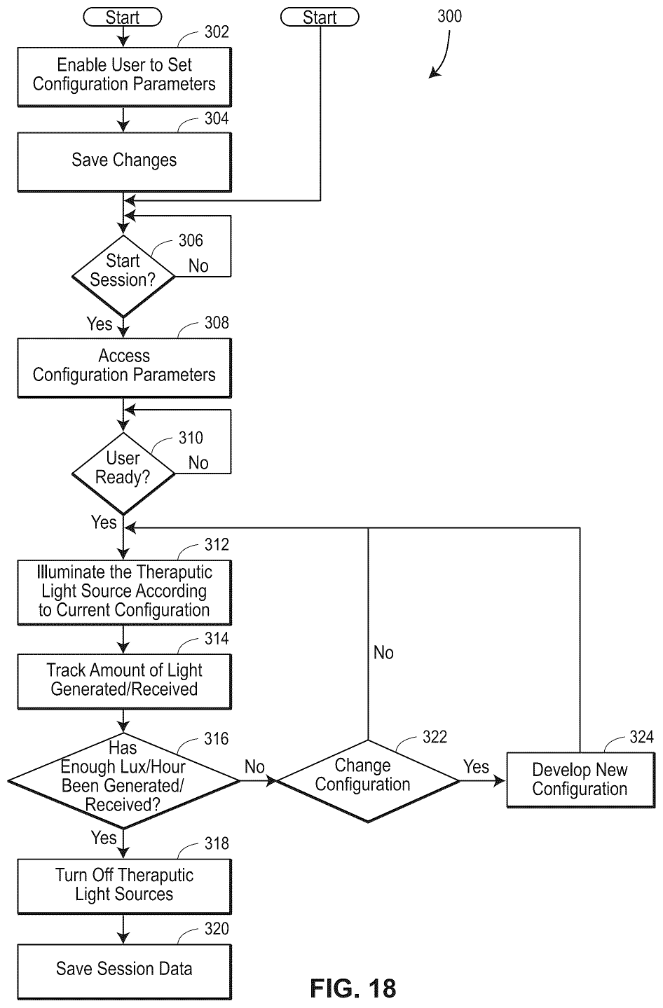

FIG. 18 illustrates a flow chart 300 of an example light therapy application or routine that may be used to implement an integrated light therapy system in a computer system. The flow chart 300 includes a configuration element 302 that may allow the user to select a type of therapy session that a user may be interested in, such as a bright light therapy session or a light therapy session associated with one or more different types of therapy. The user may type in (or otherwise enter or specify) a desired type of therapy or select one of a set of pre-established therapies. As part of this process, the element 302 may access a memory and obtain specific light therapy configuration data associated with the selected type of light therapy, such as the wavelength or wavelengths (also known as the “color”) of light to use for the therapy, the duration or amount of lux hours to be received by the user during each session, the frequency of sessions, the type of feedback to provide to the user during a light therapy session etc. Additionally or instead, the element 302 may enable a user to select, via a computer display and one or more input devices, the color of the therapeutic light, the brightness or intensity of the therapeutic light, the amount of lux to be received in each therapy session (e.g., 100, 250, 500, 1000, 5000, etc. lux hours) and one or more other configuration parameters, such as how the light for the light therapy session should be displayed (e.g., as a sun in the background), the shape(s) or location(s) of the areas of the display to be used for generating the therapeutic light for the light therapy session, whether to enable the light therapy application to change or move the areas, light sources, or amount of light used for the light therapy session during a light therapy session (e.g., based on other windows being displayed in the display screen), the time or duration of the light therapy session, etc. The element 302 may enable the user to select other configuration factors as well or instead. After the configuration process is complete, an element 304 may save the configuration to a memory to be used in later sessions.

Next, an element 306 may determine if a light therapy session should begin. If not, control is returned to the element 306 or to the configuration box 302. When a user provides an input to start a light therapy session at an element 306, an element 308 may obtain and load one or more light therapy configuration parameters from a file in the memory that specifies the particulars of the light therapy session to implement. Next, an element 310 may determine if the user is ready or in place to begin a light therapy session. The element 310 may, for example, use sensors associated with the computer system to determine if the user is properly situated in front of the display screen or other lights to be used to provide the light therapy. In a VR goggle configuration, sensors in the goggles may determine if the goggles are properly placed on the user's head and that the user is looking into the eye pieces of the goggles. When the element 310 determines that a user is properly situated or ready, an element 312 may begin to illuminate the screen or areas of the screen of the computer system and/or one or more peripheral lights associated with the computer system to provide light therapy to the user. The selection of the areas of the screen, the lights to use, the brightness and wavelengths of light to use etc. may be specified by the configuration file being used for the light therapy session.

Control may then be provided to an element 314 which tracks or records the amount of light provided to or received by the user during the current iteration or cycle of the light therapy application. The element 314 may use the specifics of the current lighting configuration to determine the amount of (e.g., brightness and surface area emitting) light being directed at or towards the user, the configured wavelength of light, one or more determinations of the distance between the light sources and the user's eyes, etc. to determine an amount of lux provided to the user during the current cycle of the light therapy application. If desired, the element 314 may use feedback from one or more sensors of the computer system to determine or fine tune the lux determination, such as whether the user's eyes are open or closed, the size of the user's pupils, where the user is looking, the distance from the light sources to the user's face, a measured amount of light incident upon the user, a measured wavelength of light incident upon the user, etc.

An element 316 may then compare the accumulated measurement of lux received by the user to the desired amount for a light therapy session and determine if the user has received the desired amount of lux hours for a session. If so, an element 318 turns off or releases (to the operating system) the light sources or areas of screen being used to provide therapeutic light and an element 320 may store the amount of light or lux, and other specifics of the therapy session in a memory. The element 318 may stop providing therapeutic light in any desired manner, such as by halting the operation of the pixels or light sources providing therapeutic light, by fading these pixels or light sources out over a period of time (e.g., 3-10 seconds) or in any other manner. If, at the element 316, not enough light has been received for a session, a block 322 determines if any configuration changes need to be made to the light emitting sources used to provide therapeutic light. Such configuration changes may change the area or areas of the screen being used to emit therapeutic light, the brightness or wavelength of light to be emitted for therapeutic light, etc. These changes may be based on or caused by actions being performed on the screen by other applications that the user is using (such as if other applications are using more or less of the display screen). Moreover, these changes may be based on feedback from the user either directly or via one of the sensors that measure phenomena about the user or the environment in which the user is located, such as if the user is squinting, the ambient light around the user, where the user is looking, etc. If such changes are to be made, an element 324 makes changes to the light emission configuration and control returns to the element 312 which then uses the current light emission configuration to provide light to the user during the next cycle. Of course, the elements 312, 314, 316, 322 and 324 may continue to operate until the element 316 determines that the desired amount of light has been provided to the user for the therapy session. Still further, any of the elements 312-324 may provide feedback to the user regarding the current therapy session, such as an ongoing ratio of therapeutic light received versus the desired amount of therapeutic light to be received during the session (e.g., a progress report of the therapy session).

Additionally, a light therapy application may provide feedback regarding one or more light therapy sessions to a user and/or to a medical provider. The application may, for example, communicate the specifics of one more light therapy sessions (including any of the data stored therefor) to a user and/or to a medical provider or consultant for the user. This feedback may be sent at the user's request or automatically via any desired means of communication, such as via an internet communications connection, a cellular phone connection, a satellite connection, etc., the circuitry for which may be part of the computer system. The feedback may be used to enable a medical provider to observe or track the times of therapeutic sessions, the number of sessions, the specifics of the light received during the sessions, etc. and may be used to track the effectiveness of the therapeutic regime as well as to determine if changes need to be made to the therapeutic regime. As an aid to such tracking, the light therapy application may provide the user with a test to track the effect of one or more sessions on the user. Such tests may be provided to the user on the same or a different computer than the computing systems that provides the light therapy and, in one example, the tests may take the form of a questionnaire with questions designed to track the user's state of mind or mental condition at various times during the application of a therapeutic regime comprising multiple therapeutic sessions. The medical provider may use the tests and data regarding the specific therapeutic sessions provided to the user to make changes to the therapeutic sessions, such as the amount of light or lux hours to be received during each session, the frequency or timing of the sessions, the wavelengths of light to use for the sessions, etc. Moreover, if desired, the light therapy application may provide reminders to the user to take medication prescribed for the user, to perform exercise or other activities, etc., to treat one or more user medical conditions, including the medical condition(s) being treated by the light therapy regime. In this manner, the user is more likely to implement a complete medical regime that includes light therapy and other medical treatment, such as taking medication, regular exercise, etc.

When implemented in software, the light therapy applications and components or routines thereof described herein may be stored in any tangible, non-transitory computer readable memory such as on a magnetic disk, a laser disk, solid state memory device, molecular memory storage device, or other storage medium, in a RAM or ROM of a computer or processor, etc. Although the example systems disclosed herein are disclosed as including, among other components, software and/or firmware executed on hardware, it should be noted that such systems are merely illustrative and should not be considered as limiting. For example, it is contemplated that any or all of these hardware, software, and firmware components could be embodied exclusively in hardware, exclusively in software, or in any combination of hardware and software. Accordingly, while the example systems described herein are described as being implemented in software executed on a processor of one or more computer devices, persons of ordinary skill in the art will readily appreciate that the examples provided are not the only way to implement such systems.

Thus, while the present invention has been described with reference to specific examples, which are intended to be illustrative only and not to be limiting of the invention, it will be apparent to those of ordinary skill in the art that changes, additions or deletions may be made to the disclosed embodiments without departing from the spirit and scope of the invention. Still further, the particular features, structures, and/or characteristics of any specific embodiment described and/or illustrated herein may be combined in any suitable manner and/or in any suitable combination with one and/or more other embodiments, including the use of selected features with or without corresponding use of other features. In addition, many modifications may be made to adapt a particular application, situation and/or material to the essential scope or spirit of the present invention. It is to be understood that other variations and/or modifications of the embodiments of the present invention described and/or illustrated herein are possible in light of the teachings herein and should be considered part of the spirit or scope of the present invention. Moreover, it will be understood that certain aspects of the invention are described herein as exemplary aspects but the invention described herein are not limited to these aspects and may not necessarily include each of these aspects.

1. A computing system comprising:
a processor and a memory having stored thereon computer-executable instructions that, when executed,
cause the computing system to administer light therapy to a user by controlling a display screen to emit light of a specific wavelength and intensity beneficial for treating a neurological condition while simultaneously enabling the user to interact with one or more non-therapeutic applications on the computing system.

2. The computing system of claim 1, wherein the computer-executable instructions, when executed, further track the duration or the intensity of the light therapy provided to the user.

3. The computing system of claim 1, further comprising a configuration application stored in the memory that, when executed on the processor, enables a user to change the configuration of the light therapy.

4. The computing system of claim 3, wherein the configuration application automatically adjusts the configuration of the light provided during a light therapy session based on feedback from one or more sensors.

5. The computing system of claim 4, wherein the configuration application adjusts one or more of the wavelength of the emitted light, the intensity of the emitted light, or a light source from which the emitted light emanates.

6. The computing system of claim 1, further comprising sensors communicatively coupled to the processor and wherein the computer-executable instructions further operate to detect the user's presence and automatically initiate or pause the light therapy session.

7. The computing system of claim 1, further comprising one or more peripheral light sources controlled by the computing system to supplement the light therapy administered through the display screen.

8. A light therapy administration device, comprising:
a computer display screen;
a computer memory;
a processor coupled to the computer display screen;
a non-light therapeutic application stored in the computer memory and executable on the processor to provide user content via at least a portion of the computer display screen; and
a light therapy application stored in the computer memory and executable on the processor to provide a light therapy session by controlling the computer display screen to emanate therapeutic light from one or more portions of the computer display screen while the non-light therapeutic application executes on the processor.

9. The light therapy administration device of claim 8, further comprising a configuration application stored in the computer memory and executable on the processor to configure the manner in which the therapeutic light emanates from the computer display screen.

10. The light therapy administration device of claim 9, wherein the configuration application enables a user to configure one or more operational parameters of the light therapy session.

11. The light therapy administration device of claim 10, wherein the one or more parameters of the light therapy session includes at least one of the temporal length of the light therapy session, the intensity of therapeutic light emitted during the light therapy session, the wavelength of therapeutic light emitted during the light therapy session, and the portion of the computer display screen used for the light therapy session.

12. The light therapy administration device of claim 8, wherein the light therapy application uses one or more fixed portions of the computer display screen for providing therapeutic light to the user during the light therapy session.

13. The light therapy administration device of claim 8, wherein the light therapy application changes the portion of the computer display screen from which therapeutic light emits during a light therapy session.

14. The light therapy administration device of claim 8, wherein the light therapy application changes the portion of the computer display screen from which therapeutic light emits during a light therapy session based on the operation of the non-light therapeutic application.

15. The light therapy administration device of claim 8, further comprising one or more sensors that measure one or more light parameters related to the therapeutic light provided to a user during a light therapy session, the one or more sensors being communicatively coupled to the processor.

16. The light therapy administration device of claim 15, wherein light therapy application uses the one or more light parameters to control the therapeutic light being emitted from the computer display screen.

17. The light therapy administration device of claim 16, wherein the one or more of the light parameters includes at least one of the intensity of the light delivered to a user during the light therapy session and the wavelength of the therapeutic light delivered to the user during the light therapy session.

18. The light therapy administration device of claim 8, further comprising one or more user feedback sensors that detect one or more user feedback parameters from the user during the light therapy session, the sensors being communicatively coupled to the processor.

19. The light therapy administration device of claim 18, wherein the one or more user feedback sensors includes a camera.

20. The light therapy administration device of claim 18, wherein the light therapy application uses the one or more user feedback parameters to determine a position of a user's eye during the light therapy session.

21. The light therapy administration device of claim 18, wherein the light therapy application uses the one or more user feedback parameters to determine a position of a user's eyelid during the light therapy session.

22. The light therapy administration device of claim 18, wherein the light therapy application uses the one or more user feedback parameters to determine a reaction of the user to the therapeutic light emitted from the computer display screen during the light therapy session.

23. The light therapy administration device of claim 18, wherein the light therapy application informs the user when a particular amount of therapeutic light has been emitted from the computer display screen or received by the user.

24. The light therapy administration device of claim 18, wherein the light therapy application automatically stops emitting therapeutic light when a particular amount of therapeutic light has been emitted from the computer display screen or received by the user.

25. A light therapy administration device, comprising:
a computer display screen;
a computer memory;
a processor coupled to the computer display screen;
a non-light therapeutic application stored in the computer memory and executable on the processor to provide user content via at least a portion of the computer display screen;
one or more additional light sources coupled to the processor to provide therapeutic light; and
a light therapy application stored in the computer memory and executable on the processor to provide a light therapy session by controlling the one or more additional light sources to emit therapeutic light while the non-light therapeutic application executes on the processor using the computer display screen.

26. The light therapy administration device of claim 25, wherein one of the one or more additional light sources is a further computer display screen.

27. The light therapy administration device of claim 25, further comprising a configuration application stored in the computer memory and executable on the processor to configure the manner in which the therapeutic light emanates from the one or more additional light sources.

28. The light therapy administration device of claim 25, wherein the configuration application enables a user to configure one or more operational parameters of the light therapy session.

29. The light therapy administration device of claim 28, wherein the one or more operational parameters of the light therapy session includes at least one of the temporal length of the light therapy session, the intensity of therapeutic light emitted during the light therapy session, and the wavelength of therapeutic light emitted during the light therapy session.

30. The light therapy administration device of claim 25, further comprising one or more sensors that measure one or more light parameters related to the therapeutic light provided to a user during a light therapy session, the one or more sensors being communicatively coupled to the processor.

31. The light therapy administration device of claim 30, wherein light therapy application uses the one or more light parameters to control the therapeutic light being emitted from the one or more additional light sources.

32. The light therapy administration device of claim 25, further comprising one or more user feedback sensors that detect one or more user feedback parameters from the user during the light therapy session, the sensors being communicatively coupled to the processor.
